(19) United States
(12) Patent Application Publication
(10) Pub. No.: US 2005/0123487 A1
(43) Pub. Date: Jun. 9, 2005

(54) Stable nonaqueous liquid reactive skin care and cleansing packaged product

(76) Inventors: Alessandro Luigi Spadini, Stamford, CT (US); Melissa Iva Katz, Weston, CT (US); David Robert Williams, Monroe, CT (US); Marcina Siciliano, New Haven, CT (US); Evan Hillman, Torrington, CT (US); Andre Puleo, Bethany, CT (US); Megan Kathleen Hurley, Middletown, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/730,709

(22) Filed: Dec. 8, 2003

(51) Int. Cl. A61K 7/11; A61K 7/06

(52) U.S. Cl. 424/47; 424/70.11

(57) ABSTRACT

A cleansing or skin treatment product is described which has one or more chambers and contains a substantially nonaqueous composition having a continuous and a discontinuous phase in at least one chamber. Components of the discontinuous phase can chemically react with each other or with water when water is blended with the nonaqueous cleansing or skin treatment composition during consumer use.

### BACKGROUND

1. Field of the Invention

The present invention relates to a liquid or viscous skin care or cleansing packaged product.

2. Background of the Invention

Conventional aqueous based liquid cleansing products have been traditionally used by consumers for personal cleansing. Frequently such products have compositions that are diluted with water to pre-solubilize or disperse the surfactant and benefit agent ingredients and are purchased by consumers in their diluted state. Consumers desiring to purchase a product containing a concentrated liquid version of such a cleansing composition, with reduced levels of water, are frequently faced with a very viscous and inconvenient to use product. Consumers also desire to purchase skin care and cleansing compositions with active ingredients that are freshly prepared and have a high degree of activity for its intended function. Concentrated nonaqueous cleansing products and compositions are known, some of which having active ingredients that are activated during use. For example, PCT publication no. WO 02/069917 to Glenn et al. published on Sep. 12, 2002 discloses an oil-in-oil emulsion that contains cosmetic actives that react with amino acid substrates where an internal oil solvates the reactive agent. U.S. publication no: 2002/0192173 to Glenn et al. published on Dec. 19, 2002 discloses an anhydrous treatment composition comprising an agent that reacts with amino acid based substrates and a solvent which solvates the reactive agent and which is water miscible. U.S. Pat. No. 6,451,327 issued to Masaaki et al. on Sep. 17, 2002 discloses a substantially nonaqueous, water-miscible skin cleansing composition containing a granulated surfactant. U.S. Pat. No. 6,569,415 issued to Orloff et al. on May 27, 2003 discloses an indicating shaving preparation product that undergoes a discernible physical or chemical change after a sufficient amount of water has been intermixed with the shaving preparation. Physical or chemical changes are described to take place in the shaving preparation itself or by disrupting encapsulated active ingredients dispersed within the shaving preparation and allowing the active ingredients to react with water or other components of the preparation. Also described by Orloff et al. is one embodiment of the product dispenser that contains two chambers that are isolated from each other and a system for blending the components together to allow them to react upon dispensing. There is no disclosure or suggestion however of unsolvated dispersed phase active ingredients that are not encapsulated in a barrier material prior to activation. Another example of a nonaqueous reactive product is provided in U.S. Pat. No. 6,274,127 issued to Schraer et al. on Aug. 14, 2001 which discloses an antiperspirant composition containing water reactive monomers in a nonaqueous base that polymerize when activated by the moisture in perspiration. A further example of a solid nonaqueous reactive product is provided in U.S. Pat. No. 3,194,736 issued to Braun on Jul. 13, 1965 which discloses a solid depilatory composition containing a dispersed sulfide ion precursor and an alkali in a waxy base that is activated by moisture applied to the skin.

Surprisingly it has been found that a skin care or cleansing product can be made containing at least one chamber holding a composition formulated with a substantially solvated or continuous phase and a substantially unsolvated or discontinuous phase where at least two components of the discontinuous phase may either chemically react with each other when blended with water or where at least one of the components may itself chemically react with the water so as to provide a unique cleansing, skin benefit, cosmetic benefit, sensory signal or a combination thereof to the user. Such a product also solves the problem of providing a product that can dispense a concentrated cleansing or skin benefit component in a convenient liquid or viscous form for consumer use.

### SUMMARY OF THE INVENTION

In one aspect of the inventive skin care or cleansing packaged product is a product including but not limited to:
a. a container having a first chamber and optionally a second chamber, the chamber(s) having an outlet;
b. a composition contained in the first chamber having a dispersed phase including a first component, the first component being capable of chemically reacting with a second component that is different from the first. Preferably the first and second components are not encapsulated in a barrier material prior to reaction or at any time;
c. a continuous phase present in the composition composed of a substantially anhydrous carrier contained in the first chamber, preferably the moisture content of the carrier is less than about 1% by wt., preferably less than about 0.9, 0.7, 0.5, 0.3, 0.1, 0.05, or 0.01% by wt. at 25 C; more preferably the carrier is immiscible in water at 25 C);
d. at least one stabilizer contained in the dispersed phase wherein the stabilizer is selected from an organophilic particle, an amphipathic compound or polymer, or a crystalline hydroxyl containing stabilizer;
e. an anionic surfactant contained in the dispersed phase in a concentration of at least 2% by wt. when the at least one stabilizer consists solely of waxy particles, amphipathic compounds or polymers, or a combination thereof;
f. an anionic surfactant in a concentration of at least 2, 3, 5, 7, 9, 10, 15, 20, or 30% by wt. when the at least one stabilizer consists solely of waxy particles, amphipathic compounds or polymers, or a combination thereof; and
g. wherein the first component is substantially unsolvated in the carrier.

Chemically reacting as used herein is defined as but is not limited to gas formation, redox reactions, lysis (e.g. hydrolysis and perhydrolysis), bond cleavage and the like; and does not include reactions that manifest themselves solely by one or more of the following: 1) color formation or color change, 2) self-polymerization and 3) exothermic or endothermic solvation processes. Chemical reactions are not excluded from the invention merely because they are accompanied by color change, self-polymerization, and exothermic or endothermic solvation processes if they also include at least one other definable chemical reaction. Substantially anhydrous as used herein means that the carrier is sufficiently free of water to prevent substantial solvation or reaction with the first component. Substantially anhydrous as used herein can also mean that the carrier contains water but that the water is isolated or otherwise prevented from solvating or reacting with the first component. Substantially unsolvated is herein defined as where the reactive component's solubility in the carrier or a solidifiable matrix (as discussed below) is less than about 5, 3, 2, 1, 0.5, or 0.1% by wt. at 25° C.

### DETAILED DESCRIPTION OF THE INVENTION

In one aspect of the inventive skin care or cleansing packaged product is a product including but not limited to:
a. a container having a first chamber and optionally a second chamber, the chamber(s) having an outlet;
b. a composition contained in the first chamber having a dispersed phase including a first component, the first component being capable of chemically reacting with a second component that is different from the first. Preferably the first and second components are not encapsulated in a barrier material prior to reaction or at any time;
c. a continuous phase present in the composition composed of a substantially anhydrous carrier contained in the first chamber, preferably the moisture content of the carrier is less than about 1% by wt., preferably less than about 0.9, 0.7, 0.5, 0.3, 0.1, 0.05, or 0.01% by wt. at 25 C; more preferably the carrier is immiscible in water at 25 C);
d. at least one stabilizer contained in the dispersed phase wherein the stabilizer is selected from an organophilic particle, an amphipathic compound or polymer, or a crystalline hydroxyl containing stabilizer;
e. an anionic surfactant contained in the dispersed phase in a concentration of at least 2, 3, 5, 7, 9, 10, 15, 20, or 30% by wt. when the at least one stabilizer consists solely of waxy particles, amphipathic compounds or polymers, or a combination thereof; and
f. wherein the first component is substantially unsolvated in the carrier.

Advantageously the product has both a first and a second chamber and the outlet of the first chamber and the outlet of the second chamber fluidly communicate with each other to allow mixing of the contents of the first and second chamber prior to deposition on the skin or hair of a user. Preferably the product has both a first and a second chamber and the outlet of the first chamber and the outlet of the second chamber do not fluidly communicate with each other whereby the mixing of the contents of the first and second chamber occurs after deposition of the contents of the first and second chamber on the skin or hair of a user. Preferably the second component is water.

In a preferred embodiment of the invention the dispersed phase in the first chamber has less than 25% by wt. of the second component and the dispersed phase in the second chamber has less than 25% by wt. of the first component. In another embodiment of the invention, a cup is releasably attached to the container and positioned for receiving the contents of the first chamber. Preferably the cup is marked with a fill line for introducing a measured amount of the second component. More preferably the cup is positioned for receiving the contents of the second chamber and the packaged product further includes a unit dose pump. Advantageously the outlet of the first chamber contains a one way valve; preferably the valve is selected from a duckbill valve, a ball valve or a slit valve (and the like).

Advantageously the at least one reactive component has a particle size range of about 0.1 to 5000μ (preferably in the range of about 0.1 to 100μ, more preferably in the range of about 0.5 to 10μ). Alternatively the lowest average particle size can be 0.01, 0.02, 0.05, 0.1, 0.2, 0.5, 1.0 or 2.0 microns and the highest average particle size can be 10, 11, 15, 20, 30, 50, 100, 110, 150, 200, 500, 1000, 2000 or 5000 microns.

Advantageously, the stabilizer may be substantially solvated in the continuous phase, (preferably where the stabilizer solubility in the carrier is greater than about 1 gm/L at 25° C. or about 5 gms/L at 25° C.). Preferably the stabilizer is selected from an organophilic particle, an amphipathic compound or polymer, or a crystalline hydroxyl containing stabilizer. More preferably the stabilizer is an organophilic particle in the particle size range of about 0.02 to 250μ and advantageously selected from a waxy particle, organophilic silica, organophilic clay, or blends thereof. In a preferred embodiment the stabilizer is an amphipathic compound or polymer with some oil soluble groups substantially solvated by the carrier and some polar groups substantially unsolvated by the carrier and is advantageously selected from polysiloxanes, polyalkylene ethers, polysaccharides, polyacrylates, or polystyrene each substituted with at least one linear or branched C8 to C24 alkyl or alkenyl chain.

In a preferred embodiment, the first component is capable of producing a gas in aqueous solution when reacted with an acid and the second component forms an acid in the presence of water. In another preferred embodiment the first component is capable of generating a peroxide compound when dissolved in water. In a further preferred embodiment the first component is capable of generating sulfide ions when reacted with an alkaline material and water.

Reactive and Anhydrous Chemistry Configurations

Useful chemistry configurations include a cosmetic composition with suspended insolubilized surfactants in a solidified matrix or a substantially nonaqueous liquid carrier. Other useful chemistry systems may include effervescent cosmetic compositions, bleaching systems, or any system that may react with each other or with water and that are compatible with the other constituents of the inventive product.

Effervescent cosmetic compositions with particular ratios of different organic acids for different sensory effects which e.g. vary with water solubility such as citric, malic, tartaric, and fumaric acids combined with carbonate or bicarbonate salts where at least one of the acid or of the carbonate/bicarbonate salt is in the discontinuous phase may be used.

Bleaching systems may be used that contain anhydrous sodium perborate and/or sodium percarbonate and the like. These materials are hydrogen peroxide donors when in contact with water—preferably where the a pH is greater than about 7.0, 7.5, 8.0, 8.5, 9.0 or 9.5.

Depilatory systems may be used that contain a precursor acid that reacts with the base when the formulation is hydrated to form the active material for removing hair from the body. These acids may include such as thiolactic acid, thioglycolic acid and other aliphatic mercapto acids and the like. These materials may be activated by ingredient(s) within the dispersed phase (such as one or more alkaline materials or precursor(s) thereof), within the continuous phase, within both phases or by a component added to the inventive composition during product use such as water or encapsulated ingredient(s) liberated during product use such as by rubbing or by two packaging chambers which during use can be broken to allow for mixing of the two phases (such as water and the inventive composition).

Other useful additions to the inventive composition include materials with exothermic heats of solution or dispersion in water (such as zeolites and the like) or materials with endothermic heats of solution or dispersion in water (such as ammonium chloride, and the like). Carbon dioxide encapsulated by any suitable solid water soluble or dispersible material such as starch or sugar or blends thereof (such as Pop Rocks™, or chemicals that react to change color upon contact with water or with each other when solvated with water such as any water soluble or dispersible colorant e.g. blue 1, yellow 5 or 10, green 3, 5, or 7; blends thereof and the like.

The inventive cosmetic composition may also include reactive ingredients which are structured with waxes, polymers, etc. to form solid forms. The inventive cosmetic compositions may also contain an oil, an emulsifier, an organoclay, and 10% or more dispersed solids by weight, or may contain materials that impart a cooling sensation on the skin such as menthol and derivatives and the like.

Structurants and Stabilizers

Products according to the invention may also include compositions containing in-use water structurants (such as lauric acid and trihydroxystearin); and stabilizers such as self-orienting/structuring organophilic particles which impart rheological elasticity (yield stress) such as organically modified clays (chemically reacted with fatty quaternium compounds for hydrophobicity) based on Hectorite, Bentonite, or synthetic clays such as Hydrotalcite that are available from Rheox/Elementis (Hightstown, N.J.), Southern Clay (Gonzales, Tex.), and SUD Chemie (Munich, Germany) respectively. Other useful components include elastomers such as those with silicone or nonsilicone backbones with different crosslinking groups, such as phenylated and polyvinyl linkages and the like. Specific useful elastomers include DC 9040 available from Dow Corning (Midland, Mich.), GE SFE818 available from General Electric (Waterford, N.Y.), Belsil RG 100 available from Wacker (Munich, Germany), and KSG 21 available from ShinEtsu (Tokyo, Japan). Such elastomers can act as stabilizers for the inventive composition.

Filler or oil absorbing particulates which are insoluble in the continuous phase may be advantageously used. Useful materials in this category include ultra-fine materials such as mica, talc, titanium dioxide, silica and starch. Finely powdered silicone polymers such as KSP 100 available from ShinEtsu may be used. Hollow and/or low density materials such as starch spheres (e.g. Natrasorb-W) available from National Starch (Bridgewater, N.J.), polymeric spheres (e.g. Expancel available from Akzo Nobel (Duluth, Ga.); and borosilicate glass spheres (e.g. Luxsil available from PQ corporation (Philadelphia, Pa.). These materials can act as stabilizers for the inventive composition.

Hydrophobic polymeric gellants may be used to adjust the viscosity of the continuous phase. Useful materials include Krayton Gel (e.g. Krayton D-1101 available from Krayton Polymers Corporation (Houston, Tex.); Hydrophobized PVP copolymers (e.g. Ganex series available from ISP Products Inc. (Wayne, N.J.); Silicone polymers (e.g. DC2-1491) available from Dow Corning (Midland, Mich.), Acrylate polymers/copolymers (e.g. poly(sodium acrylate)) available from Rohm & Haas company (Philadelphia, Pa.); and Silicone-acrylate polymers (e.g. SA 70, VS 70, and VS80 available from 3M company (St. Paul, Minn.). These materials can act as stabilizers for the inventive composition.

Crystalline hydroxyl-containing stabilizers including ethoxylated fatty alcohols (e.g. Neodol from Shell (Houston, Tex.) and trihydroxystearin (e.g. Thixcin-R from Rheox (Hightstown, N.J.) may be advantageously used.

Waxy materials such as organic waxes; silicone waxes, silicone-acrylate waxes, fatty amides may be useful in the inventive composition. Other useful components include high melt point hydrocarbons (e.g. having a melting point of greater than 55 C such as petrolatum. These materials can act as stabilizers for the inventive composition.

Polymers that form water gels and travel to emulsion interfaces upon contact with water are also useful as stabilizers in the inventive composition such as Pemulen® (high molecular weight, cross linked copolymers of acrylic acid and a hydrophobic comonomer) and Carbopol® (high molecular weight homo- and copolymers of acrylic acid, optionally crosslinked with various substituents such as polyalkenyl polyethers) available from Noveon Chemicals (Cleveland, Ohio) and the like; Glyceril Polyacrylates (e.g. Lubrajel series from ISP (Wayne, N.J.)).

Surfactants

Soaps

The inventive product may contain a soap in its continuous or discontinuous phase. The term “soap” is used herein in its popular sense, i.e., the alkali metal or alkanol ammonium salts of alkane- or alkene monocarboxylic acids. Sodium, potassium, mono-, di- and tri-ethanol ammonium cations, or combinations thereof, are suitable for purposes of this invention. The soaps useful herein are the well known alkali metal salts of alkanoic or alkenoic acids having about 8 to 50 carbon atoms, preferably about 12 to about 22 carbon atoms. They may also be described as alkali metal carboxylates of alkyl or alkene hydrocarbons having about 12 to about 22 carbon atoms.

Soaps may be made by the classic kettle boiling process or modern continuous soap manufacturing processes wherein natural fats and oils such as tallow or coconut oil or their equivalents are saponified with an alkali metal hydroxide using procedures well known to those skilled in the art. Alternatively, the soaps may be made by neutralizing fatty acids, such as lauric (C₁₂), myristic (C₁₄), palmitic (C₁₆), or stearic (C₁₈) acids with an alkali metal hydroxide or carbonate.

Anionic Surfactants

One or both of the continuous or discontinuous phases may also contain non-soap anionic surfactants. The anionic surfactant (which may comprise about 0 to 45% by wt. of total composition; 0 to 25% in the continuous phase and 0 to 45% in the discontinuous phase) may be, for example, an aliphatic sulfonate, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or an aromatic sulfonate such as alkyl benzene sulfonate, and the like.

The anionic may also be an alkyl sulfate (e.g., C₁₂-C₁₈ alkyl sulfate) or alkyl ether sulfate (including alkyl glyceryl ether sulfates), and the like. Among the alkyl ether sulfates are those having the formula:
RO(CH₂CH₂O)ₙSO₃M
wherein R is an alkyl or alkenyl having 8 to 18 carbons, preferably 12 to 18 carbons, n has an average value of greater than 1.0, preferably between 2 and 3; and M is a solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, and acyl isethionates, and the like.

Sulfosuccinates may be monoalkyl sulfosuccinates having the formula:
R⁴O₂CCH₂CH(SO₃M)CO₂M;
amido-MEA sulfosuccinates of the formula
   R⁴CONHCH₂CH₂O₂CCH₂CH(SO₃M)CO₂M
wherein R⁴ ranges from C₈-C₂₂ alkyl and M is a solubilizing cation;
amido-MIPA sulfosuccinates of formula
   RCONH(CH₂)CH(CH₃)(SO₃M)CO₂M
where M is as defined above.

Also included are the alkoxylated citrate sulfosuccinates; and alkoxylated sulfosuccinates such as the following: 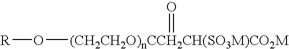
wherein n=1 to 20; and M is as defined above.

Sarcosinates are generally indicated by the formula RCON(CH₃)CH₂CO₂M, wherein R ranges from C₈ to C₂₀ alkyl and M is a solubilizing cation.

Taurates are generally identified by formula
R²CONR³CH₂CH₂SO₃M
wherein R² ranges from C₈-C₂₀ alkyl, R³ ranges from C₁-C₄ alkyl and M is a solubilizing cation.

Another class of anionics are carboxylates such as follows:
R—(CH₂CH₂O)ₙCO₂M
wherein R is C₈ to C₂₀ alkyl; n is 0 to 20; and M is as defined above.

Another carboxylate which can be used is amido alkyl polypeptide carboxylates such as, for example, Monteine LCQ® by Seppic.

Another surfactant which may be used are the C₈-C₁₈ acyl isethionates. These esters are prepared by reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

Acyl isethionates, when present, will generally range from about 0.5-15% by weight of the total composition. Preferably, this component is present from about 1 to about 10%.

The acyl isethionate may be an alkoxylated isethionate such as is described in U.S. Pat. No. 5,393,466, Titled “Fatty Acid Esters Of Polyalkoxylated Isethionic Acid” issued Feb. 28, 1995 to Ilardi et al., hereby incorporated by reference into the subject application. This compound has the general formula: 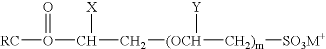
wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

Zwitterionic and Amphoteric Surfactants

One or both of the continuous or discontinuous phases may also contain zwitterionic/amphoteric surfactants. Zwitterionic surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. A general formula for these compounds is: 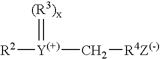
wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Examples of such surfactants include:
4-[N,N-di(2-hydroxyethyl)-N-octadecylammonio]-butane-1 carboxylate;
5-[S-3-hydroxypropyl-S-hexadecylsulfonio]-3-hydroxypentane-1-sulfate;
3-[P,P-diethyl-P-3,6,9-trioxatetradexocylphosphonio]-2-hydroxypropane-1-phosphate;
3-[N,N-dipropyl-N-3-dodecoxy-2-hydroxypropylammonio]-propane-1-phosphonate;
3-(N,N-dimethyl-N-hexadecylammonio)propane-1-sulfonate;
3-(N,N-dimethyl-N-hexadecylammonio)-2-hydroxypropane-1-sulfonate;
4-[N,N-di(2-hydroxyethyl)-N-(2-hydroxydodecyl)ammonio]-butane-1-carboxylate;
3-[S-ethyl-S-(3-dodecoxy-2-hydroxypropyl)sulfonio]-propane-1-phosphate;
3-[P,P-dimethyl-P-dodecylphosphonio]-propane-1-phosphonate; and
5-[N,N-di(3-hydroxypropyl)-N-hexadecylammonio]-2-hydroxy-pentane-1-sulfate.

Amphoteric detergents which may be used in this invention include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula: 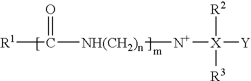
where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Suitable amphoteric detergents within the above general formula include simple betaines of formula: 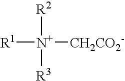
and amido betaines of formula: 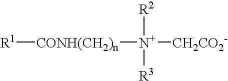
where m is 2 or 3.

In both formulae R¹, R² and R³ are as defined previously. R¹ may in particular be a mixture of C₁₂ and C₁₄ alkyl groups derived from coconut so that at least half, preferably at least three quarters of the groups R¹ have 10 to 14 carbon atoms. R² and R³ are preferably methyl. A suitable betaine is cocoamidopropyl betaine.

A further possibility is that the amphoteric detergent is a sulphobetaine of formula 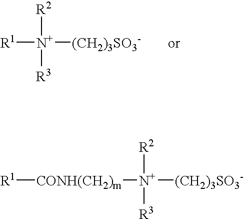
where m is 2 or 3, or variants of these in which —(CH₂)₃SO⁻₃ is replaced by

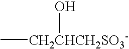

In these formulae R¹, R² and R³ are as discussed previously.

Amphoacetates and diamphoacetates are also intended to be covered in possible zwitterionic and/or amphoteric compounds which may be used, especially C8-C20 amphoacetates or mixtures thereof, and the like. A suitable amphoacetate is sodium laurylamphoacetate.

The amphoteric/zwitterionic surfactant, when used, generally comprises about 0 to 30%, preferably about 5 to 20% by weight, more preferably about 3 to 10% of the composition. 0 to 15% in the continuous phase and 0 to 30% in the discontinuous phase).

A preferred surfactant system of the invention comprises the following: anionic surfactant (e.g. alkali metal alkyl ethersulfate), about 0-50%; amphoteric surfactant (e.g. alkyl betaine or alkyl amphoacetate), about 0-20% based on the total composition.

The surfactant system may also optionally comprise a nonionic surfactant. The nonionic which may be used includes in particular the reaction products of compounds having a hydrophobic group and a reactive hydrogen atom, for example aliphatic alcohols, acids, amides or alkyl phenyls with alkylene oxides, especially ethylene oxide either alone or with propylene oxide. Specific nonionic detergent compounds are alkyl (C₆-C₂₂) phenyls-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxides, and the like.

The nonionic may also be a sugar amide, such as a polysaccharide amide. Specifically, the surfactant may be one of the lactobionamides described in U.S. Pat. No. 5,389,279 titled “Compositions comprising nonionic glycolipid surfactants” issued on Feb. 14, 1995 to Au et al. which is hereby incorporated by reference or it may be one of the sugar amides described in U.S. Pat. No. 5,009,814 titled “Use of n-polyhydroxyalkyl fatty acid amides as thickening agents for liquid aqueous surfactant systems” issued on Apr. 23, 1991 to Kelkenberg, hereby incorporated into the subject application by reference.

Other surfactants which may be used are described in U.S. Pat. No. 3,723,325 to Parran Jr. and alkyl polysaccharide nonionic surfactants as disclosed in U.S. Pat. No. 4,565,647 titled “Foaming surfactant compositions”, issued on Jan. 21, 1986 to Lienado, both of which are also incorporated into the subject application by reference.

Preferred alkyl polysaccharides are alkylpolyglycosides of the formula
R²O(CₙH₂ₙO)ₜ(glycosyl)ₓ
wherein R² is selected from the group consisting of alkyl, alkylphenyl, hydroxyalkyl, hydroxyalkylphenyl, and mixtures thereof in which alkyl groups contain from about 10 to about 18, preferably from about 12 to about 14, carbon atoms; n is 0 to 3, preferably 2; t is from 0 to about 10, preferably 0; and x is from 1.3 to about 10, preferably from 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4- and/or 6-position, preferably predominantly the 2-position.

The nonionic comprises about 0 to 40% by wt. in each phase of the composition, preferably about 0 to 15% by wt. 0 to 20% in the continuous phase and 0 to 20% in the discontinuous phase)

Occlusive Emollients

One way of moisturizing is to reduce the rate of water loss from the stratum corneum (skin surface) by depositing an occlusive emollient on the skin surface which prevents water evaporation. Another technique is to add hygroscopic nonocclusive substances (humectants), which will retain water to the stratum corneum, making water available to the skin surface thereby producing the desired cosmetic effect. Nonocclusive emollients also function by improving the lubricity of the skin. Both occlusive and nonocclusive emollients as well as mixtures thereof are operative in the present invention and may be present in either or both the continuous or discontinuous phases. Examples of occlusive emollients include, lanolin and its derivatives, long chain esters, waxes, saturated and unsaturated fatty alcohols, conditioning oils and extracts, phospholipids, sterols, ceramides and silicones. The following occlusive emollients may optionally be found in the compositions of the invention.

Vegetable oils: Arachis oil, castor oil, cocoa butter, coconut oil, corn oil, cotton seed oil, olive oil, palm kernel oil, rapeseed oil, safflower seed oil, sesame seed oil and soybean oil, and the like.

Esters: Butyl myristate, cetyl palmitate, decyloleate, glyceryl laurate, glyceryl ricinoleate, glyceryl stearate, glyceryl isostearate, hexyl laurate, isobutyl palmitate, isocetyl stearate, isopropyl isostearate, isopropyl laurate, isopropyl linoleate, isopropyl myristate, isopropyl palmitate, isopropyl stearate, propylene glycol monolaurate, propylene glycol ricinoleate, propylene glycol stearate, and propylene glycol isostearate, and the like.

Animal Fats: acetylated lanolin alcohols, lanolin, lard, mink oil and tallow, and the like.

Other examples of occlusive emollients include mineral oil, petrolatum, silicone oil such as dimethyl polysiloxane, lauryl and myristyl lactate, fatty acid oils, triglycerides, and the like.

The occlusive emollient is generally used in an amount from about 0 to 70%, preferably about 5 to 40% by wt. of the phase in which it is found in. Generally, it should comprise no more than 70% of such phase. A portion of the emollient may be present in the form of solid or semi-solid beads. The beads are optionally used in an amount from about 0.01, 0.05, 0.1, 0.5 or 1.0% by wt. to about 5, 10, 15 or 20% by wt.

Nonocclusive Emollients

Some examples of nonocclusive emollients are liquid water-soluble polyols, glycerin, propylene glycol, sorbitol, polyethylene glycol, ethoxylated/propoxylated ethers of methyl glucose (eg., methyl gluceth-20) and ethoxylated/propoxylated ethers of lanolin alcohol e.g., Solulan-75). Some other preferred moisturizers are the nonoclusive liquid water soluble polyols and the essential amino acid compounds found naturally in the skin. Other preferred nonocclusive moisturizers are compounds found to be naturally occurring in the stratum corneum of the skin, such as sodium pyrrolidone carboxylic acid, lactic acid, urea, L-proline, guanidine and pyrrolidone. Examples of other nonocclusive moisturizers include hexadecyl, myristyl, isodecyl isopropyl esters of adipic, lactic, oleic, stearic, isostearic, myristic or linoleic acids, as well as many of their corresponding alcohol esters (sodium isostearoyl-2 lactylate, sodium capryl lactylate), hydrolyzed protein and other collagen-derived proteins, aloe vera gel and acetamide MEA. Other examples of both types occlusive and nonocclusive emollients are disclosed in “Emollients—a Critical Evaluation,” by J. Mausner Cosmetics & Toiletries, May 1981, incorporated herein by reference.

In addition, the continuous or discontinuous phases of the compositions of the invention may include optional ingredients as follows:

Sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer); all of which are useful in enhancing the appearance or cosmetic properties of the product.

The compositions may further comprise antimicrobials such as 2-hydroxy-4,2′4′ trichlorodiphenylether (DP300), quaternary ammonium compounds; preservatives such as dimethyloldimethylhydantoin (Glydant XL1000), parabens, sorbic acid etc.

The compositions may also comprise coconut acyl mono- or diethanol amides and the like as suds boosters.

Antioxidants such as, for example, butylated hydroxytoluene (BHT) may be used advantageously in amounts of about 0.01% or higher if appropriate.

Cationic conditioners which may be used include Polyquaternium-10, Quatrisoft LM-200, Polyquaternium-24, Merquat Plus 3330, Polyquaternium 39, Ucare polymer JR-400, Jaguar® type conditioners and the like.

Another optional ingredient which may be added are the deflocculating polymers such as are taught in U.S. Pat. No. 5,147,576 titled “Liquid Detergent Composition In The Form Of Lamellar Droplets Containing A Deflocculating Polymer”, issued on Sep. 15, 1992 to Montague, hereby incorporated by reference.

Other ingredients which may be included are exfoliants such as polyoxyethylene beads, silica particles, walnut shells and apricot seeds, and the like, pH and viscosity adjusters may be optionally used to e.g. adjust the pH of the separate phases prior to being combined into the inventive product. Such suitable pH adjusters may include citric acid, glycolic acid, lactic acid, other alpha or beta hydroxy acids, and the like.

Product Form

The anhydrous compositions according to the present invention may be provided in any suitable physical form provided that they can be amply intermixed together by the consumer prior or simultaneous to application to skin and hair, for example as low to moderate viscosity liquids. These compositions may be produced by procedures well known to the skilled artisan. The cosmetic compositions can be used in various ways as other known compositions in the art including but not limited to various rinse-off and leave-on applications such as hair shampoos, skin cleansers, skin lotions, hair conditioners, hair dyes, hair straighteners, hair bleaches, styling sprays, hair mousses, two-in-one shampoos, fabric softeners, lotions, hair waving, and the like.

The anhydrous compositions should be stored in a manner that separates the reactive components for over the shelf life of the product while providing ample protection from the atmosphere/environment. This can be accomplished in a preferred embodiment via a single package or single chamber arrangement where a water reactive ingredient or two or more reactive ingredients are held in an unsolvated state in an anhydrous carrier. In another preferred embodiment, a two package or two-chamber arrangement may be provided wherein one package or chamber contains the first component and the other package or chamber contains the second component. A number of mechanisms can be used to ensure that the two components are mixed either immediately prior or during use by the consumer including, but not limited to:
1. A dual or multi-chamber package whereby the first component is stored within one chamber and the second component within the other chamber and wherein the contents of all the chambers are intermixed via or after dispensing for use, e.g., dispensing through the same orifice, dispensing through separate orifices followed by mixing within one's hands, dispensing through separate orifices followed by mixing upon application to hair or skin, or combinations thereof
2. Multiple packages wherein the first component is stored within one of the packages, e.g., bottle, vial, sachet etc., and the separate second component is stored within a separate package, e.g., bottle, vial or sachet, and whereby the volume of one of the packages is sufficiently large enough to enable the addition of the contents of the remaining packages, via pouring, dispensing etc., to enable intermixing of each of the separate components prior to use,
3. Pouring the contents of both packages or compartments into one's hands which can be mixed by rubbing the hands together or via massaging onto the skin or hair, and
4. A multichambered package whereby the first component is stored within one chamber and the second component within the other chamber and wherein the chambers are separated by temporary ‘barriers’ that are removed or compromised prior to use to enable mixing, e.g., deformable barrier(s), breakable of barrier(s), removable barrier(s) etc Conversely, either of the first or second compositions can be placed within shear sensitive (or other induced release) capsules which are dispersed within the other respective composition and which rupture (or other induced release) prior or during use.

The invention will now be described in greater detail by way of the following non-limiting examples. The examples are for illustrative purposes only and not intended to limit the invention in any way.

Except in the examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

All percentages in the specification and examples are intended to be by weight unless stated otherwise.

### EXAMPLE 1

An inventive cleansing product having two chambers and a mixing conduit communicating with the outlet of each chamber may be prepared according to table 1. The composition is useful as a shampoo and for oily skin cleansing and provides a stimulating effect to the skin via the evolution of carbon dioxide bubbles during cleansing.

TABLE 1

| | | | Total |
|---|---|---|---|
| | | | Dispensed |
| | Chamber | Chamber | Concentration |
| Component | A | B | (w/w) |
| Water | 0% | 80% | 40% |
| Water Soluble | 34.5% | 15% | 24.75% |
| Anhydrous Fluid such as | | | |
| Propylene Glycol or | | | |
| Anhydrous | | | |
| Glycerin | | | |
| Non Polar Oil such as | 5% | 0% | 2.5% |
| Isohexadecane, or | | | |
| Polyisobutene. | | | |
| Ethoxylated Fatty | 10% | 0% | 5% |
| Alcohol | | | |
| Emollient Ester | 3% | 0% | 1.5% |
| Fatty Amide | 2% | 0% | 1% |
| Hyrocarbon/Silicone | 1% | 0% | 0.5% |
| Wax such as paraffin or | | | |
| Shinetsu | | | |
| KP 100. | | | |
| Anhydrous Suspended | 10% | 0% | 5% |
| or Solubilzed Anionic | | | |
| Surfactant | | | |
| Powder | | | |
| Anhydrous Suspended | 1% | 0% | 0.5% |
| or Solubilized Amphoteric | | | |
| Surfactant Powder | | | |
| Hydrophilic Structuring | 1% | 2% | 1.5% |
| Polymer such as Pemulen | | | |
| or Carbopol. | | | |
| Sodium Bicarbonate | 15% | 0% | 7.5% |
| Citric Acid | 15% | 0% | 7.5% |
| Fragrance | 2% | 1% | 1.5% |
| Menthol or derivatives | 0.5% | 0% | 0.25% |
| Benzoyl Peroxide | 0% | 2% | 1% |
| TOTAL | 100% | 100% | 100% |

### EXAMPLE 2

An inventive cleansing product having two chambers and a mixing conduit communicating with the outlet of each chamber may be prepared according to table 2. The product is useful as a lotion that provides a stimulating effect to the skin via the evolution of carbon dioxide bubbles upon the addition of water.

TABLE 2

| | | | Total |
|---|---|---|---|
| | | | Dispensed |
| | Chamber | Chamber | Concentration |
| Components | A | B | (w/w) |
| Water | 0% | 100% | 50% |
| Non-Polar Oil | 32% | 0% | 16% |
| Ethoxylated Fatty | 10% | 0% | 5% |
| Alcohol | | | |
| Emollient Ester** | 4% | 0% | 2% |
| Fatty Amide** | 2% | 0% | 1% |
| Organic/Silicone Wax | 1% | 0% | 0.5% |
| Liquid Emulsifier** | 5% | 0% | 2.5% |
| Suspended water | 2% | 0% | 1% |
| gellant such as pemulen | | | |
| or carbopol. | | | |
| Organic modified clay | 15% | 0% | 7.5% |
| such as Rheox Bentone | | | |
| 38V | | | |
| Hydrophobic Polymeric | 2% | 0% | 1% |
| Structurant | | | |
| Sodium Bicarbonate | 15% | 0% | 7.5% |
| Citric Acid | 15% | 0% | 7.5% |
| Fragrance | 2% | 0% | 1% |
| TOTAL | 100% | 100% | 100% |

### EXAMPLE 3

An inventive moisturizing cleansing product having two chambers and a mixing conduit communicating with the outlet of each chamber may be prepared according to table 3. In addition to a stimulating effect to the skin from carbon dioxide evolution, the product has color changing properties when blended with water.

TABLE 3

| | | | Total |
|---|---|---|---|
| | | | Dispensed |
| | Chamber | Chamber | Concentration |
| Components | A | B | (w/w) |
| Water | 0% | 100% | 50% |
| Sunflower Seed Oil | 27.99% | 0% | 13.995% |
| Neodol 45 (ethoxylated | 10% | 0% | 5% |
| fatty alcohol) | | | |
| Bentone 38 ISD GEL | 15% | 0% | 7.5% |
| (Organoclay) | | | |
| Tauranol I78 (Sodium Co- | 10% | 0% | 5% |
| coyl Isethionate Powder) | | | |
| Brij 93 Vej | 5% | 0% | 2.5% |
| Sodium Bicarbonate | 15% | 0% | 7.5% |
| Citric Acid | 15% | 0% | 7.5% |
| Green #3 | 0.01% | 0% | 0.005% |
| Fragrance | 2% | 0% | 1% |
| TOTAL | 100% | 100% | 100% |

### EXAMPLE 4

An inventive wash-off moisturizing cleansing product having two chambers and a mixing conduit communicating with the outlet of each chamber may be prepared according to table 4. The product has a stimulating effect to the skin from carbon dioxide evolution when blended with water.

TABLE 4

| | | | Total |
|---|---|---|---|
| | | | Dispensed |
| | Chamber | Chamber | Concentration |
| Components | A | B | (w/w) |
| Water | 0% | 100% | 50% |
| Sunflower Seed Oil | 30% | 0% | 15% |
| Bentone 38 ISD GEL | 15% | 0% | 7.5% |
| (Organoclay) | | | |
| Tauranol I78 (Sodium | 15% | 0% | 7.5% |
| Cocoyl Isethionate | | | |
| Powder) | | | |
| Brij 93 Vej | 8% | 0% | 4% |
| Sodium Bicarbonate | 15% | 0% | 7.5% |
| Citric Acid | 15% | 0% | 7.5% |
| Fragrance | 2% | 0% | 1% |
| TOTAL | 100% | 100% | 100% |

### EXAMPLE 5

An inventive moisturizing cleansing product having two chambers and a mixing conduit communicating with the outlet of each chamber may be prepared according to table 5. The product has a stimulating effect to the skin from carbon dioxide evolution when blended with water.

TABLE 5

| | | | Total |
|---|---|---|---|
| | | | Dispensed |
| | Chamber | Chamber | Concentration |
| Components | A | B | (w/w) |
| Water | 0% | 100% | 50% |
| Non-Polar Oil | 20% | 0% | 10% |
| Fatty Alcohol | 10% | 0% | 5% |
| Ethoxylated Fatty | 4 | 0% | 2% |
| Alcohol | | | |
| Emolient Ester | 4 | 0% | 2% |
| Fatty Amide | 2% | 0% | 1% |
| Organic/Silicone Wax | 3% | 0% | 1.5% |
| Anhydrous | 8% | 0% | 4% |
| Suspended Anionic | | | |
| Surfactant Powder | | | |
| Anhydrous | 2% | 0% | 1% |
| Suspended Amphoteric | | | |
| Surfactant Powder | | | |
| Organic Clay | 15% | 0% | 7.5% |
| Hydrophobic | 2% | 0% | 1% |
| Polymeric Structurant | | | |
| Sodium Bicarbonate | 13% | 0% | 6.5% |
| Citric Acid | 13% | 0% | 6.5% |
| Fragrance | 2% | 0% | 1% |
| Hydrophilic Polymeric | 2% | 0% | 1% |
| Structurant such as | | | |
| Pemulen | | | |
| or Carbopol | | | |
| TOTAL | 100% | 100% | 100% |

### EXAMPLE 6

An inventive lotion product having two chambers and a mixing conduit communicating with the outlet of each chamber may be prepared according to table 6. The product has a stimulating effect to the skin from carbon dioxide evolution when blended with water.

TABLE 6

| | Chamber | Chamber | Concentration |
|---|---|---|---|
| Components | A | B | (w/w) |
| Water | 0% | 100% | 50% |
| Sunflower Seed Oil | 35% | 0% | 17.5 |
| Bentone 38 ISD GEL | 15% | 0% | 7.5% |
| (Organoclay) | | | |
| Brij 93 Vej | 10% | 0% | 5% |
| Sodium Bicarbonate | 15% | 0% | 7.5% |
| Citric Acid | 15% | 0% | 7.5% |
| Fragrance | 5% | 0% | 2.5% |
| Silicone oil such as | 5% | 0% | 2.5% |
| dimethicone or phenyl | | | |
| trimethicone | | | |
| TOTAL | 100% | 100% | 100% |

### EXAMPLE 7

An inventive water activated bleaching product for bleaching body hair having two chambers and a mixing conduit communicating with the outlet of each chamber may be prepared according to table 7.

TABLE 7

| | Chamber | Chamber | Concentration |
|---|---|---|---|
| Components | A | B | (w/w) |
| Water | 0% | 100% | 50% |
| Sunflower Seed Oil | 35% | 0% | 17.5% |
| Bentone 38 ISD GEL | 15% | 0% | 7.5% |
| (Organoclay) | | | |
| Brij 93 Vej | 15% | 0% | 7.5% |
| Neodol 45 | 15% | 0% | 7.5% |
| Sodium Carbonate | 0% | 5% | 2.5% |
| Anhydrous Sodium | 15% | 0% | 7.5% |
| Perborate or Sodium | | | |
| Percarbonate powder | | | |
| Fragrance | 5% | 0% | 2.5% |
| TOTAL | 100% | 100% | 100% |

### EXAMPLE 8

An inventive shear thinning viscous cleansing product having two chambers and a mixing conduit communicating with the outlet of each chamber may be prepared according to table 8.

TABLE 8

| | Chamber | Chamber | Concentration |
|---|---|---|---|
| Components | A | B | (w/w) |
| Water | 0% | 100% | 50% |
| Sunflower Seed Oil | 10% | 0% | 5% |
| Bentone 38 ISD GEL | 35% | 0% | 7.5% |
| (Organoclay) | | | |
| Brij 93 Vej | 5% | 0% | 2.5% |
| Neodol 45 | 15% | 0% | 7.5% |
| Sodium Bicarbonate | 15% | 0% | 7.5% |
| Citric Acid | 15% | 0% | 7.5% |
| Fragrance | 5% | 0% | 2.5% |
| TOTAL | 100% | 100% | 100% |

### EXAMPLE 9

An inventive water-activated depilatory product may be prepared according to table 9.

TABLE 9

| | | | Total |
|---|---|---|---|
| | | | Dispensed |
| | Chamber | Chamber | Concentration |
| Components | A | B | (w/w) |
| Water | 0% | 100% | 50% |
| Sunflower Seed Oil | 29.5% | 0% | 14.75% |
| Bentone 38 ISD GEL | 15% | 0% | 7.5% |
| (Organoclay) | | | |
| Brij 93 Vej | 5% | 0% | 2.5% |
| Neodol 45 (Ethoxylated | 20% | 0% | 10% |
| Fatty Alcohol) | | | |
| Thiolactic Acid | 13% | 0% | 6.5% |
| (Powder) | | | |
| Sodium Bicarbonate | 5% | 0% | 2.5% |
| Calcium Hydroxide | 12%* | 0% | 6%* |
| (Fine Powder) | | | |
| Fragrance | 0.5% | 0% | 0.25% |
| TOTAL | 100% | 100% | 100% |

*Calcium Hydroxide is used at a level sufficient to provide a pH of 10.5 to 12.5 in a saturated aqueous solution of the depilatory composition dispensed by the user.

As alternative preferred embodiments of the invention, each of the exemplified inventive cleansing products of examples 1 to 9 may be further divided among 3 or more separate chambers whose outlets optionally communicate with a mixing conduit and where the contents of at least two of the chambers are blended with each other before consumer use. In a third alternative arrangement of the exemplified inventive cleansing products of examples 1 to 9, the contents of chambers A and B, absent the water, are combined in a single chamber. The contents of this single chamber are then combined with water just prior to or during consumer use. The criteria for combining components in a single chamber is that the stability of the composition be maintained and that no appreciable decomposition or unintended chemical reaction take place until consumer use. No appreciable decomposition or unintended chemical reaction means that the contents of any one chamber do not substantially react with each other until dispersed or dissolved in water, preferably where no more than about 10, 5, 2, 1, 0.5 or 0.1% by wt. of any component of the contents of a chamber undergoes a chemical reaction when stored at 50 C for 60 days prior to being dispensed.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

1. A skin care or cleansing packaged product, comprising:
a. a container having a first chamber and optionally a second chamber, the chamber(s) having an outlet;
b. a composition contained in the first chamber having a dispersed phase including a first component, the first component being capable of chemically reacting with a second component that is different from the first;
c. a continuous phase present in the composition composed of a substantially anhydrous carrier contained in the first chamber;
d. at least one stabilizer contained in the dispersed phase wherein the stabilizer is selected from an organophilic particle, an amphipathic compound or polymer, or a crystalline hydroxyl containing stabilizer;
e. an anionic surfactant contained in the dispersed phase in a concentration of at least 2% by wt. when the at least one stabilizer consists solely of waxy particles, amphipathic compounds or polymers, or a combination thereof; and
f. wherein the first component is substantially unsolvated in the carrier.

2. The packaged product of claim 1 wherein the product has both a first and a second chamber and the outlet of the first chamber and the outlet of the second chamber fluidly communicate with each other to allow mixing of the contents of the first and second chamber prior to deposition on the skin or hair of a user.

3. The packaged product of claim 1 wherein the product has both a first and a second chamber and the outlet of the first chamber and the outlet of the second chamber do not fluidly communicate with each other whereby the mixing of the contents of the first and second chamber occurs after deposition of the contents of the first and second chamber on the skin or hair of a user.

4. The packaged product of claim 2 wherein the dispersed phase in the first chamber has less than 25% by wt. of the second component and the dispersed phase in the second chamber has less than 25% by wt. of the first component.

5. The packaged product of claim 1 wherein a cup is releasably attached to the container and positioned for receiving the contents of the first chamber.

6. The packaged product of claim 5 wherein the cup is marked with a fill line for introducing a measured amount of the second component.

7. The packaged product of claim 1 where the second component is water.

8. The packaged product of claim 5 wherein the cup is positioned for receiving the contents of the second chamber.

9. The packaged product of claim 1 further comprising a unit dose pump.

10. The packaged product of claim 1 wherein the outlet of the first chamber contains a one way valve.

11. The packaged product of claim 10 wherein the valve is selected from a duckbill valve, a ball valve or a slit valve.

12. The packaged product of claim 1 wherein the at least one reactive component has a particle size range of about 0.1 to 5000μ.

13. The packaged product of claim 1 wherein the stabilizer is substantially solvated in the continuous phase.

14. The packaged product of claim 1 wherein the stabilizer is an organophilic particle in the particle size range of about 0.02 to 250μ.

15. The packaged product of claim 1 wherein the stabilizer is selected from a waxy particle, organophilic silica, organophilic clay, or blends thereof.

16. The packaged product of claim 1 wherein the stabilizer is an amphipathic compound or polymer with some oil soluble groups substantially solvated by the carrier and some polar groups substantially unsolvated by the carrier.

17. The packaged product of claim 1 where the stabilizer is an amphipathic polymer selected from polysiloxanes, polyalkylene ethers, polysaccharides, polyacrylates, or polystyrene each substituted with at least one linear or branched C8 to C24 alkyl or alkenyl chain.

18. The packaged product of claim 1 wherein the first component is capable of producing a gas in aqueous solution when reacted with an acid and the second component forms an acid in the presence of water.

19. The packaged product of claim 1 wherein the first component is capable of generating sulfide ions when reacted with an alkaline material and water.

20. The packaged product of claim 1 wherein the first component is capable of generating a peroxide compound when dissolved in water.
